SEQUENCE LISTING (4 sequences)

SEQ ID NO: 1 - protein, 414 aa, Homo sapiens [sequence omitted]

SEQ ID NO: 2 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 3 - protein, 435 aa, Homo sapiens
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 4 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

(19) United States
(12) Patent Application Publication
(10) Pub. No.: US 2015/0133340 A1
(43) Pub. Date: May 14, 2015

(54) METHOD OF DETERMINING TERATOGENIC RISK

(71) Applicant: Hoffmann-La Roche Inc., Little Falls, NJ (US)

(72) Inventors: Eric Chiao, Chatham, NJ (US); Sei Kameoka, Montclair, NJ (US); Kyle L. Kolaja, Montclair, NJ (US)

(21) Appl. No.: 14/381,430

(22) PCT Filed: Mar. 4, 2013
(86) PCT No.: PCT/EP2013/054244

§ 371 Date: Aug. 27, 2014

Related U.S. Application Data
(60) Provisional application No. 61/607,622, filed on Mar. 7, 2012.
(60) Provisional application No. 61/668,489, filed on Jul. 6, 2012.

(51) Int. Cl. G01N 33/50

(52) U.S. Cl. CPC G01N 33/5073; G01N 33/5014; G01N 33/5023; G01N 2500/10
USPC 506/10; 435/29; 435/7.1; 435/6.13

(57) ABSTRACT

The present invention relates to a method of determining the risk of drug induced teratogenicity using pluripotent stem cells.

### FIELD OF THE INVENTION

The present invention relates to a method of determining the risk of drug induced teratogenicity using human pluripotent stem cells.

### BACKGROUND OF THE INVENTION

Improved in vitro systems for predicting drug-induced toxicities are needed by the pharmaceutical and biotechnology industries to decrease late-stage drug attrition. Specifically, there is a need for predictive high-throughput in vitro models that employ human cells and can reduce our reliance on animal studies.

One of the most devastating adverse drug effects that can occur is teratogenicity. Unfortunately, predicting whether a drug has the potential to cause fetal harm in humans is extremely difficult and primarily depends on the use of pregnant animal models. Currently, due to a lack of robust alternative methods, the FDA requires several phases of animal studies to assess the risk of reproductive harm (Bailey, G. P., L. D. Wise, et al. (2009), “Pre- and postnatal developmental toxicity study design for pharmaceuticals.” Birth Defects Research Part B: Developmental and Reproductive Toxicology 86(6): 437-445). However, the dependence on animal systems runs the risk of failing to identify human teratogens such as thalidomide, that are harmful to human fetal development, but not other animal species such as rodents (Shuey, D. and J. H. Kim (2011), “Overview: developmental toxicology-new directions.” Birth Defects Research Part B: Developmental and Reproductive Toxicology, 92(5): 381-383).

Pluripotent stem cells (PSCs) can be grown indefinitely in a dish while maintaining a “blank” or “undifferentiated” stem cell state. Then, by changing the growth conditions, the cells can be directed to “differentiate” into all the tissues of the human body. During the “differentiation” process, the cells are regulated by the normal genetic programs that control fetal development. A method for characterizing compounds for teratogenic risk using pluripotent mouse embryonic stem (ES) cells has been pioneered by the laboratory of Hans Spielman (Spielmann, H., I. Pohl, et al. (1997), “The embryonic stem cell test (EST), an in vitro embryotoxicity test using two permanent mouse cell lines: 3T3 fibroblasts and embryonic stem cells.” In vitro Toxicology 10: 119-127). This method, referred to as the Embryonic Stem Cell Test, or EST, involves differentiating mouse ES cells for ten days until beating cardiomyocytes can be observed. The teratogenic risk is assessed by the use of a biostatistical prediction model that compares the concentrations of drug that exhibits 50% inhibition differentiation and 50% cytotoxicity of the mouse ES cells and a mouse differentiated fibroblast line (Seiler, A. E. and H. Spielmann (2011), “The validated embryonic stem cell test to predict embryotoxicity in vitro.” Nat Protoc 6(7): 961-78). However, this method suffers from many limitations such as the relatively long length of the assay, the labor-intensive methods required for differentiating the cells, the qualitative nature of scoring “beating” cardiomyocytes, and moderate accuracy of the biostatistical prediction model (Marx-Stoelting, P. E. Adriaens, et al. (2009), “A review of the implementation of the embryonic stem cell test (EST). The report and recommendations of an ECVAM/ReProTect Workshop.” Altern Lab Anim 37(3): 313-28). Furthermore, the mouse EST fails to accurately detect known human teratogens such as thalidomide.

Due to these limitations, many independent labs have sought to develop an improved in vitro method for assessing teratogenic risk. Most of these efforts have been aimed at increasing the assay complexity, based on the assumption that only a more complex in vitro system would provide adequate predictivity of the vast biological complexity of in vivo gestation. For example, attempts to improve the mouse EST have included employing toxicogenomic analysis to the standard cardiomyocyte differentiation protocol (Hewitt, M., C. M. Ellison, et al. (2010), “Integrating (Q) SAR models, expert systems and read-across approaches for the prediction of developmental toxicity.” Reproductive Toxicology 30(1): 147-160; van Dartel, D. A. M., J. L. A. Pennings, et al. (2010), “Monitoring Developmental Toxicity in the Embryonic Stem Cell Test Using Differential Gene Expression of Differentiation-Related Genes.” Toxicological Sciences 116(1): 130-139; Pennings, J. L. A., D. A. M. van Dartel, et al. (2011), “Gene set assembly for quantitative prediction of developmental toxicity in the embryonic stem cell test.” Toxicology 284(1-3): 63-71; van Dartel, D. A. M. and A. H. Piersma (2011). “The embryonic stem cell test combined with toxicogenomics as an alternative testing model for the assessment of developmental toxicity.” Reproductive Toxicology 32(2): 235-244); adding additional differentiated cell types such as endothelial cells or bone cells to the assay (Festag, M., B. Viertel, et al. (2007), “An in vitro embryotoxicity assay based on the disturbance of the differentiation of murine embryonic stem cells into endothelial cells. II. Testing of compounds.” Toxicology in Vitro 21(8): 1631-1640; Buesen, R., E. Genschow, et al. (2009), “Embryonic stem cell test remastered: comparison between the validated EST and the new molecular FACS-EST for assessing developmental toxicity in vitro.” Toxicol Sci 108(2): 389-400; zur Nieden, N. I., L. A. Davis, et al. (2010), “Comparing three novel endpoints for developmental osteotoxicity in the embryonic stem cell test.” Toxicology and Applied Pharmacology 247(2): 91-97); or assessing changes in metabolites secreted into the media during stem cell differentiation (West, P. R., A. M. Weir, et al. (2010), “Predicting human developmental toxicity of pharmaceuticals using human embryonic stem cells and metabolomics.” Toxicology and Applied Pharmacology 247(1): 18-27). Although some of these methods have resulted in an increase in predictivity, this has often been at the expense of assay throughput.

In contrast to the efforts mentioned above, the novel method described herein employs a seemingly counter-intuitive approach: shortening the stem cell differentiation to 2.5 days and examining mesendoderm lineage markers as the sole endpoint. Therefore this new approach interrogates a much earlier, transient stage of embryo development. Using human pluripotent stem cells, we demonstrate that this method achieves superior throughput and predictivity compared to existing methods.

### SUMMARY OF THE INVENTION

The present application provides a method of assessing the teratogenic risk of a compound comprising:
(1) contacting the compound with pluripotent stem cells that are being differentiated into mesendoderm;
(2) detecting the progression of embryonic development by measuring the expression of one or more developmentally regulated markers such as Sox17, EOMES or T-brachyury;
(3) determining the IC50 concentration of the compound which results in 50% inhibition of the protein expression of the one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed.

### BRIEF DESCRIPTION OF THE DRAWINGS

FIG. 1. Known compounds demonstrated to be either teratogens or non-teratogens in vivo.

FIG. 2. A time course showing the H9 human embryonic stem cell line differentiated for 3 days as described in the Protocol Methods. At each day indicated, the cells were fixed and stained with antibodies specific to either Sox17, EOMES or T-brachyury. The DNA stain DAPI was used as a counterstain to mark cell nuclei.

FIG. 3. Panel A: Experimental design graphically depicts the 3 day differentiation protocol used in the present invention. Panel B: An example of a teratogen treatment demonstrates the dose-dependent reduction of Sox17 staining.

FIG. 4. Co-expression of the cell lineage markers Sox17, EOMES (mesendoderm markers) and OCT4 (a marker of pluripotent cells and neural lineages). Panel A shows the overlapping expression of Sox17 and EOMES after 3 days of differentiation, marking the mesendodermal lineage. Panel B shows the nuclear staining of Sox17 and OCT4 are largely mutually exclusive. Panels C and D illustrate teratogen induced relative reduction of Sox17 and increase in OCT4 staining, suggesting that relative expression of cell lineage markers other than Sox17 or EOMES may be used as measures of teratogenic risk.

FIG. 5. An example 96-well plate layout for testing unknown compounds for teratogenic risk. The numbers in boxes refer to the compound in a certain concentration. For example 1.1=compound 1 in concentration 1, 2.1=compound 2 in concentration 1. In addition, P=positive control (with a known teratogen) and D=DMSO (the negative control).

FIG. 6. The table depicts the experimentally determined Sox17 inhibition IC50 for 75 compounds known to be either teratogenic or non-teratogenic in vivo. Using these data, we were able to establish an IC50 cut-off of 20 uM that could distinguish teratogenic from non-teratogenic compounds with an accuracy of 92%.

FIG. 7. The table shows the predictive value calculated from the results in FIG. 6 based on IC50 values of either 5 uM or 20 uM. The exact IC50 cut-off used by an individual should be chosen based on their tolerance of the predicted rate of false positives versus false negatives.

### DETAILED DESCRIPTION OF THE INVENTION

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “optional” or “optionally” as used herein means that a subsequently described event or circumstance may, but need not, occur, and that the description includes instances where the event or circumstance occurs and instances in which it does not.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “pluripotent stem cell” as used herein means and includes the ability of a cell to differentiate into cell types of all three lineages or germ layers (viz. endoderm, ectoderm, and mesoderm). The term multipotent has a meaning understood in the art, and includes the ability of a cell to differentiate into multiple cell types. It is also understood that multipotent cells may be more restricted in their ability to differentiate than pluripotent cells. The term induced stem cells (“iSCs”), as used herein, refer to induced pluripotent stem cells (“iPSCs”) or to induced multipotent stem cells (“iMSCs”). At times, the term “iPS” or “iPS cell” may be used instead of “iPSC”; similarly, at times the term “iMS” or “iMS cell” may be used instead of “iMSC”. The methods and compositions described herein that are applicable to iPSCs are also applicable to iSCs and iMSCs.

The term “measuring the protein expression” means determining the quantity of protein produced (e.g., in the well of cells by either direct methods such as antibody staining and quantifying the specific fluorescence of the antibody stained cells or indirectly by measuring the amount of mRNA encoding the protein).

The term “IC 50” as used herein means the concentration of a test compound that results in 50% inhibition of the measured differentiation marker relative to an untreated control.

The term “developmentally regulated markers” means genes whose expression change during development.

The term the “mesendodermal markers” means genes whose expression has been determined to be found in the mesoderm cell lineage. Examples of mesendodermal markers useful in this invention are Sox 17, EOMES, and T-brachyury.

The term “teratogenic risk” means the calculated risk or probability that a compound will be teratogenic in vivo. Similarly, the term “non-teratogenic risk” means the calculated risk or probability that a compound will not be teratogenic in vivo.

The term “Sox17” refers to the gene or gene product encoded by SRY-box 17 (SOX17, gene ID 64321, the gene product of the human homologue is exemplified in SEQ ID NO:1).

The term “EOMES” refers to the gene or gene product encoded by eomesodermin (EOMES, gene ID 8320, the gene product of the human homologue is exemplified in SEQ ID NO:2).

The term “T-brachyury” refers to gene or gene product encoded by T, brachyury homolog (mouse) (T, gene ID 6862 the gene product of the human homologue is exemplified in SEQ ID NO:3).

The term OCT4 refers to gene or gene product encoded by POU5F1 POU class 5 homeobox 1 (POU5F1, gene ID: 5460 the gene product of the human homologue is exemplified in SEQ ID NO:4).

The term “high throughput” as used herein means an assay design that allows easy screening of multiple samples simultaneously including capacity for robotic manipulation. Another desired feature of high throughput assays is an assay design that is optimized to reduce reagent usage, or minimize the number of manipulations in order to achieve the analysis desired. Examples of assay formats include 96-well or 384-well plates. It is well known in the art that as miniaturization of plastic molds and liquid handling devices are advanced, or as improved assay devices are designed, greater numbers of samples may be performed using the design of the present invention. In one embodiment, the cells are cultured and analyzed in the micro-titer plates containing a plurality of wells such as 96- or 386-well plates.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The present application provides a method of assessing the teratogenic risk of a compound comprising:
(1) contacting the compound with pluripotent stem cells that are being differentiated into mesendoderm;
(2) detecting the progression of embryonic development by measuring the protein expression of one or more developmentally regulated markers;
(3) determining the IC50 concentration of the compound which results in 50% inhibition of the protein expression of the one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed.

In particular embodiments, the protein expression of one or more mesendodermal markers are measured.

In more particular embodiments, the protein expression of one or more of the mesendodermal markers measured are selected from the group consisting of Sox 17, EOMES, and T-brachyury.

In more specific embodiments, the present application provides a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of the one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed.

In particular embodiments, the protein expression of at least one mesendodermal marker measured is Sox 17; and in more particular the protein expression of SOX17 is the only mesendodermal marker measured.

In other particular embodiments, the protein expression of at least one mesendodermal marker measured is EOMES; and in more particular the protein expression of EOMES is the only mesendodermal marker measured.

In other particular embodiments, the protein expression of at least one mesendodermal marker measured is T-brachyury; and in more particular the protein expression of T-brachury is the only mesendodermal marker measured.

In other particular embodiments, the protein expression of SOX17 and EOMES are the only mesendodermal markers measured.

In other particular embodiments, the protein expression of SOX17 and T-brachyury are the only mesendodermal markers measured.

In particular embodiments, the pluripotent stem cells are of primate origin.

In particular embodiments, the pluripotent stem cells are from an embryonic stem cell source.

In other particular embodiments, the pluripotent stem cells are from an induced pluripotent stem cell source.

In particular embodiments, the teratogenic risk is assessed by quantitating changes in the protein expression of markers by immuno-histochemistry.

In other particular embodiments, the teratogenic risk is assessed by quantitating changes in the protein expression of markers by flow cytometry.

In other particular embodiments, the teratogenic risk is assessed by quantitating changes in the protein expression of markers by fluorescence microscopy.

In other particular embodiments, the teratogenic risk is assessed by quantitating changes in the protein expression of markers by quantitating changes in the mRNA levels encoding the protein markers.

In particular embodiments, step (1) of the method of the present invention is performed after 40-60% of said pluripotent stem cells are differentiated into mesendoderm.

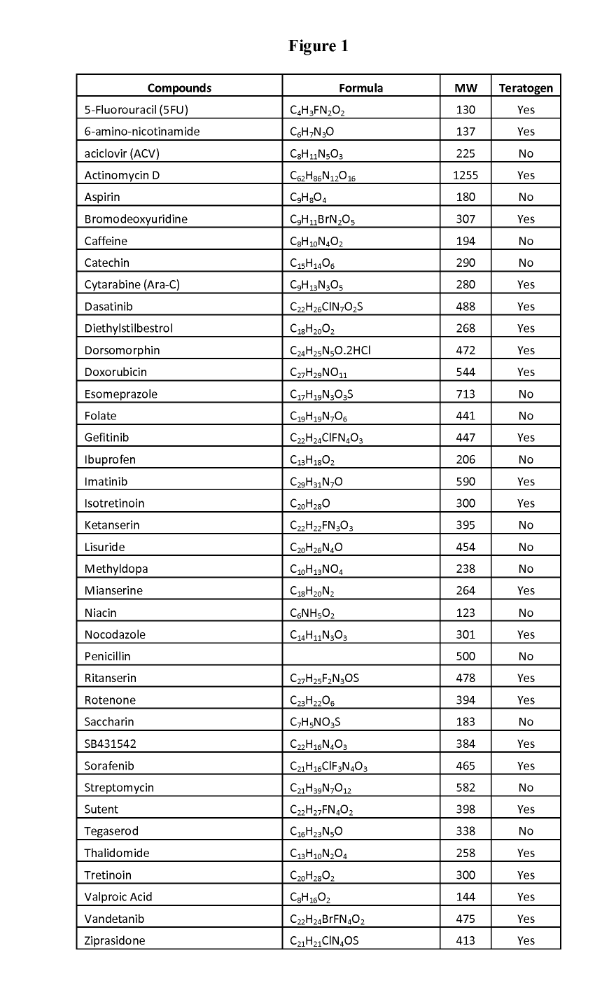

In particular embodiments, the one or more compounds with known teratogenic risks in step (4) are selected from the group consisting of known teratogens listed in FIG. 1.

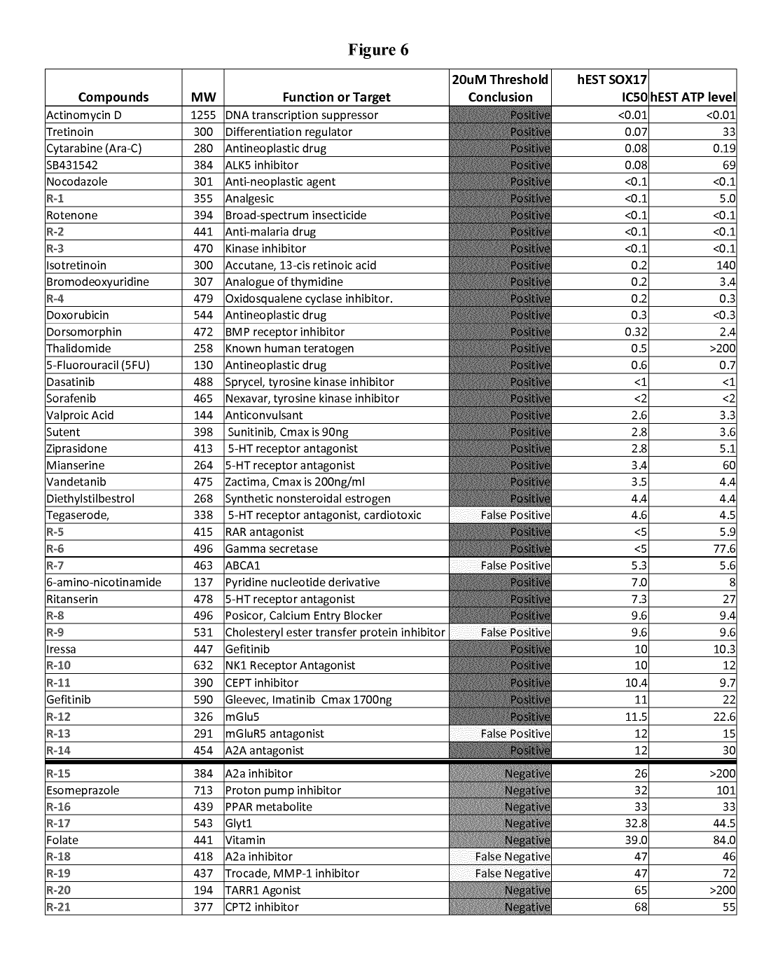

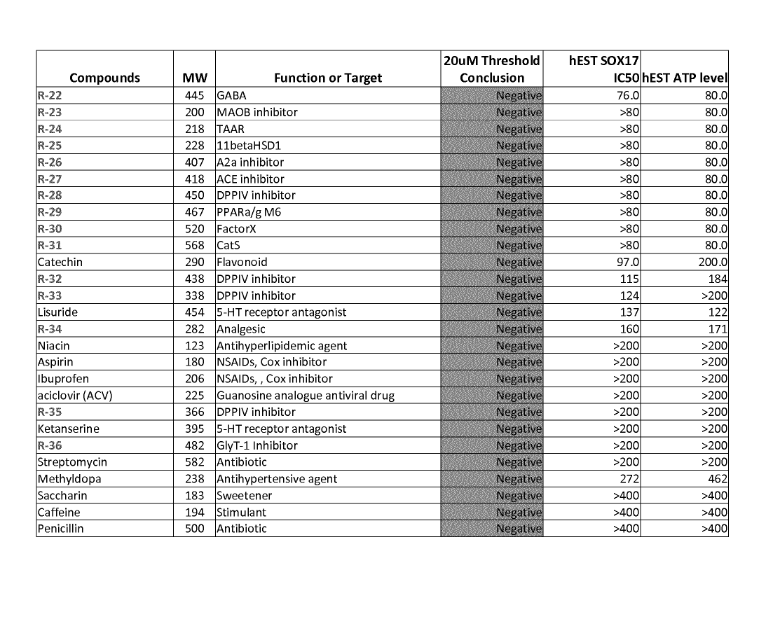

In other particular embodiments, the one or more compounds with known teratogenic risks in step (4) are selected from the group consisting of known teratogens listed in FIG. 6.

In particular embodiments, the one or more compounds with known non-teratogenic risks in step (4) are selected from the group consisting of known teratogens listed in FIG. 1.

In other particular embodiments, the one or more compounds with known non-teratogenic risks in step (4) are selected from the group consisting of known teratogens listed in FIG. 6.

In more particular embodiments, the one or more compounds with known teratogenic risks in step (4) are selected from the group consisting of: actinomycin D, tretinoin, cytarabine, nocodazole, rotenone, tretinoin, isotretinoin, bromodeoxyuridine, doxorubicin, dorsomorphin, thalidomide, 5-Fluorouracil, dasatinib, sorafenib, valproic acid, sunitinib, ziprasidone, mianserine, vandetanib, diethylstilbestrol, 6-amino-nicotinamide, ritanserin, and gefitinib.

In further particular embodiments, the one or more compounds with known teratogenic risks in step (4) are selected from the group consisting of: thalidomide, mianserine, and ritanserin.

In particular embodiments, the one or more compounds with known non-teratogenic risks in step (4) are selected from the group consisting of: esomeprazole, folate, catechin, lisuride, niacin, aspirin, ibuprofen, acyclovir, ketanserine, streptomycin, methyldopa, saccharin, caffeine, penicillin, and tegaserod.

In more particular embodiments, the one or more compounds with known non-teratogenic risks in step (4) are selected from the group consisting of: folate and methyldopa.

In more specific embodiments, the one or more compounds with known teratogenic risks in step (4) are selected from the group consisting of: thalidomide, mianserine, and ritanserin and the one or more compounds with known non-teratogenic risks are selected from the group consisting of folate and methyldopa.

In one embodiment said method is an in vitro method.

In one embodiment there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed.

In one embodiment, the pluripotent stem cells are of primate origin.

In one embodiment, the pluripotent stem cells are embryonic stem cells.

In one embodiment, the pluripotent stem cells are H9 human embryonic stem cells.

In one embodiment, the pluripotent stem cells are induced pluripotent stem cells.

In one embodiment, at least one of the mesendodermal markers is SOX17.

In one embodiment, at least one of the mesendodermal markers is EOMES.

In one embodiment, at least one of the mesendodermal markers is T-brachyury.

In one embodiment, the protein expression of SOX17 is the only mesendodermal marker measured.

In one embodiment, the protein expression of SOX17 and EOMES are the only mesendodermal markers measured.

In one embodiment, the protein expression of SOX17 and T-brachyury are the only mesendodermal markers measured.

In one embodiment, the protein expression is measured by immuno-histochemistry.

In one embodiment, the protein expression is measured by flow cytometry.

In one embodiment, the protein expression is measured by fluorescence microscopy. In one embodiment, there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed,
   wherein step (1) is performed after 40-60% of said pluripotent stem cells are differentiated into mesendoderm.

In one embodiment, there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed,
   wherein the one or more compounds with known teratogenic risks in step (4) are selected from the group consisting of: actinomycin D, tretinoin, cytarabine, nocodazole, rotenone, tretinoin, isotretinoin, bromodeoxyuridine, doxorubicin, dorsomorphin, thalidomide, 5-Fluorouracil, dasatinib, sorafenib, valproic acid, sunitinib, ziprasidone, mianserine, vandetanib, diethylstilbestrol, 6-amino-nicotinamide, ritanserin, and gefitinib.

In one embodiment, there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed,
   wherein the one or more compounds with known teratogenic risks in step (4) are selected from the group consisting of: thalidomide, mianserine, and ritanserin.

In one embodiment, there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed,
   wherein the one or more compounds with known non-teratogenic risks in step (4) are selected from the group consisting of: esomeprazole, folate, catechin, lisuride, niacin, aspirin, ibuprofen, acyclovir, ketanserine, streptomycin, methyldopa, saccharin, caffeine, penicillin, and tegaserod.

In one embodiment, there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed,
   wherein the one or more compounds with known non-teratogenic risks in step (4) are selected from the group consisting of: folate and methyldopa.

In one embodiment, there is provided a method of assessing the teratogenic risk of a compound comprising:
(1) contacting the compound with pluripotent stem cells that are being differentiated into mesendoderm;
(2) measuring the protein expression of one or more mesendodermal markers selected from the group consisting of Sox17, EOMES, and T-brachyury;
(3) determining the IC50 concentration of the compound which results in 50% inhibition of the protein expression of said one or more markers; and
(4) comparing the IC50 concentration determined in step (3) with the IC50 concentrations of: (a) one or more compounds with known teratogenic risks and (b) one or more compounds with known non-teratogenic risks, to establish a teratogenic risk of the compound being assessed,
   wherein the one or more compounds with known teratogenic risks in step (4) are selected from the group consisting of: thalidomide, mianserine, and ritanserin; and
   the one or more compounds with known non-teratogenic risks are selected from the group consisting of folate and methyldopa.

### EXAMPLES

### General Method of Determining Teratogenic Rick

The method of the present invention assesses the risk of drug induced teratogenicity by comparing the levels of mesendoderm formation in treated versus control pluripotent stem cells undergoing directed differentiation. Therefore, one must first establish the optimal conditions for directing the differentiation of the human pluripotent stem cells towards mesendoderm. As a proof of concept, an initial validation was performed using the H9 human embryonic stem cell line using the protocol described in the Examples herein.

### Optimizing the Baseline Differentiation

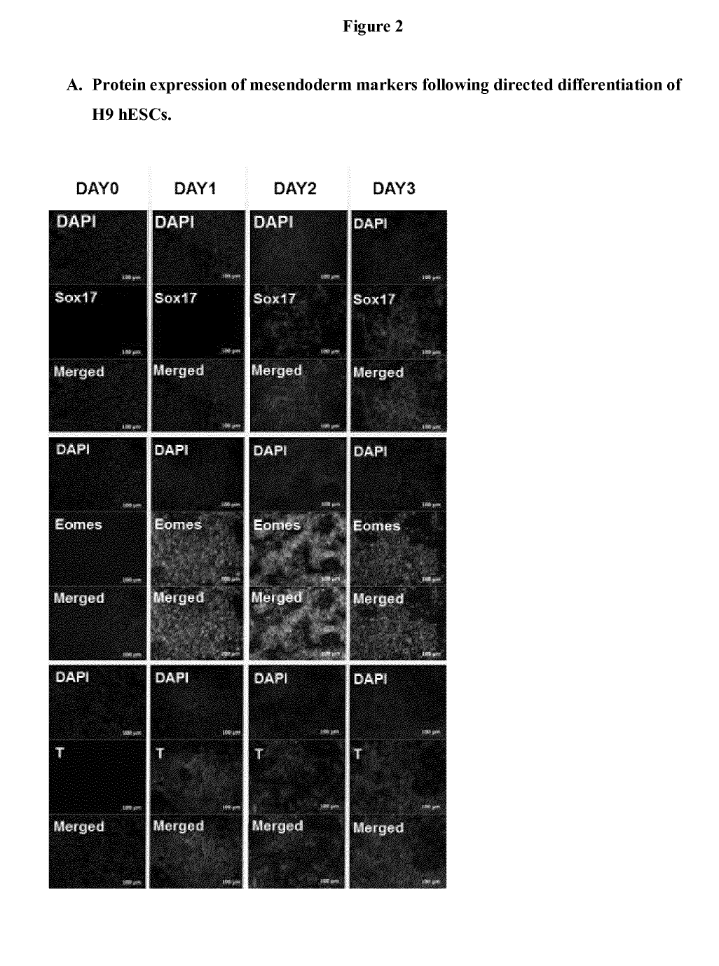

First, the optimal initial cell plating density and concentration of growth factors that reproducibly achieved 40-60% mesendoderm formation in 3 days of differentiation was identified (see FIG. 2). With minor protocol optimizations of the ideal concentrations and duration of growth factor treatments to generate mesendoderm, a person skilled in the art could adapt this method for use with most human pluripotent cell lines. Although 40-60% differentiation was chosen as the starting point for these studies, since the method relies on comparing the ratio of mesendoderm formation in drug treated samples versus control samples, the exact percent mesendoderm cells should not be critical as long as the efficiency is consistent from well to well.

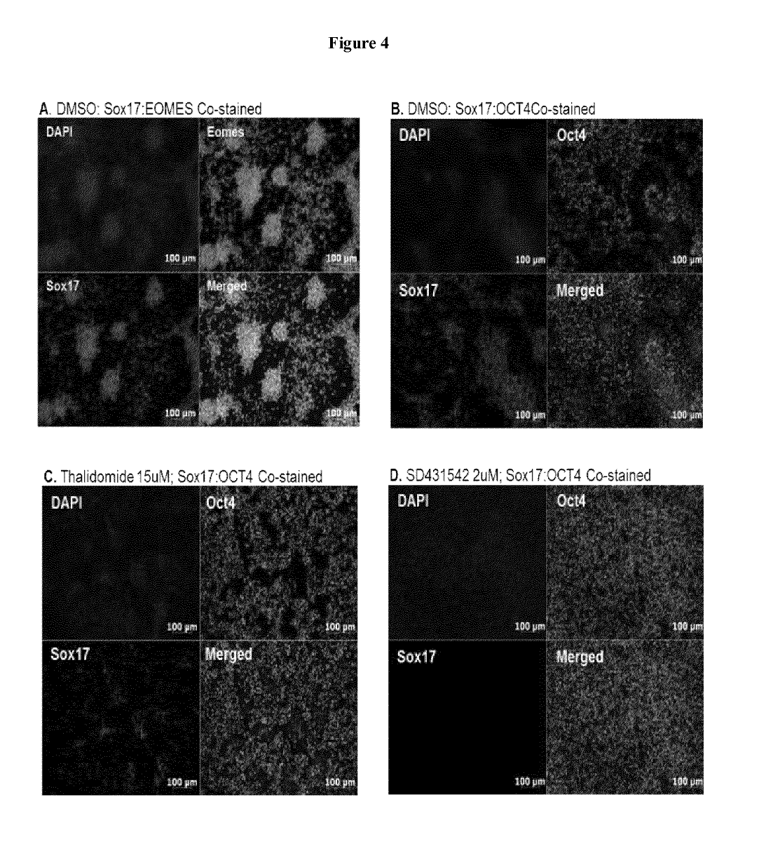

During the course of the initial validation experiments, a variety of lineage markers, antibodies, and quantitation methods were examined. Those experiments showed that the co-expressed mesendoderm lineage markers Sox17, T-brachyury and EOMES were informative for monitoring developmental disruptions. One would also predict that the quantiation of other markers that label the Sox17/EOMES/T-brachyury negative cells, such as OCT4 as shown in FIG. 4 could similarly be used to quantitate the disruption of the signal transduction pathways guiding the directed differentiation.

Establishing the Threshold Concentration for in vitro/in vivo Correlation

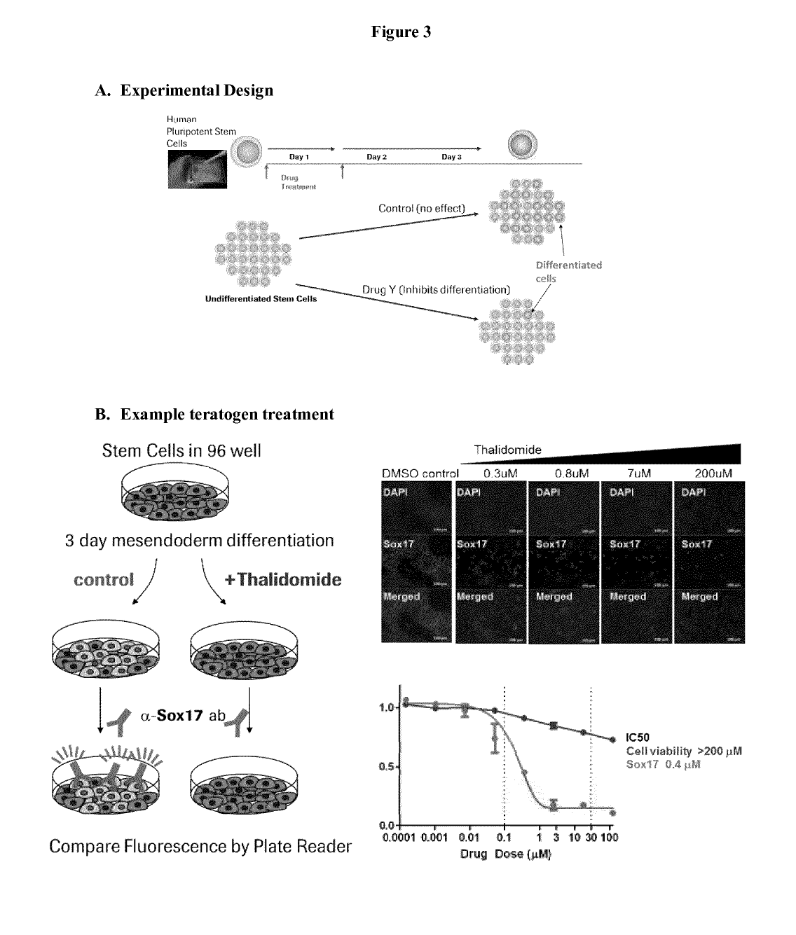

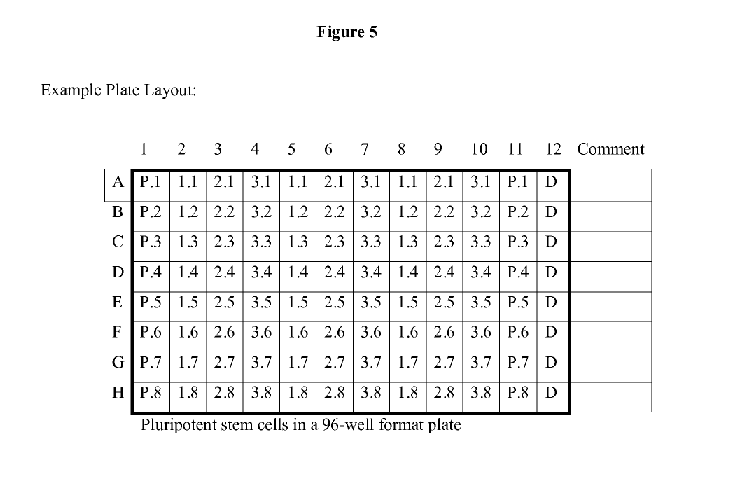

After optimizing conditions for the directed differentiation, the dose-response relationship that permits stratification of known teratogenic compounds from compounds known to not cause fetal harm is established. For the initial studies we used a panel of 30 commercially available compounds and 40 proprietary compounds with known in vivo effects. Individual wells containing cells being differentiated towards mesendoderm were treated on day 1 and day 2 (see FIG. 3). On a single plate, increasing concentrations of each compound were applied to a unique well. In addition, each plate contained wells treated with compounds known to be teratogenic as a positive control and also wells treated with only solvent as a negative control. (See example plate-map in FIG. 5).

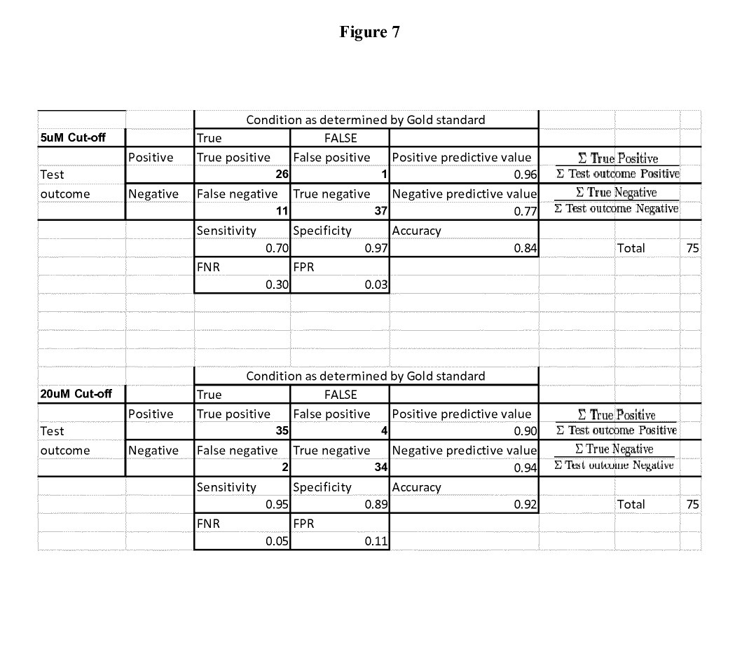

At the conclusion of the directed differentiation, the levels of mesendoderm lineage markers were quantitated. For the proof of principle studies described herein, the expression of the lineage markers in the cells was measured at the protein level by immune-staining, followed by quantification by either flow cytometry analysis, fluorescence microscopy or a using a plate reader. From these values, the concentration of each test compound that resulted in 50% inhibition of the measured differentiation marker relative to the DMSO control was calculated (the IC50) for each test compound. Examining IC50 values of the compounds with known to be either teratogenic or non-teratognic in vivo allows one to establish the IC50 values that distinguish teratogenic compounds from non-teratogenic compounds. For the H9 cell line examined (below) during the validation, it was found that a concentration of 20 uM provided >90% accuracy for predicting risk of teratogencity (see tables in FIGS. 6 and 7).

### Adapting the Assay for High-Throughput Screening

Once the desired threshold for measuring teratogenic risk is established using the pluripotent cell line of interest, the assay can be employed for higher-throughput risk assessments. Compounds can be tested in triplicate at a single concentration that correlates to a level of acceptable predictivity.

### Specific Protocol & Method For The Compounds Tested

### Basic Reagents

### Reagents

16% Formaldehyde Solution, 10×10 ml ampule, Thermo, #28908
30% Albumin Solution from bovine serum, Sigma, catalog #A9576-50 ML
100 ml Donkey Serum, Millipore, #530-100 ML
Human SOX17 NL557 Affinity Purified Polyclonal Ab, Goat IgG for immunohistochemistry R&D systems, Inc. #NL1924R
hESC-qualified Matrigel, 5 ml *LDEV-Free, BD Bioscience, #354277, extracellular matrix
Recombinant Human/Mouse/Rat Activin A, R&D Systems, Inc. #338-AC-005
Recombinant Human Wnt-3a, R&D Systems, Inc. #5036-WN-010
EmbryoMax® ES Cell Qualified Fetal Bovine Serum, 500 ml, Millipore, #ES009B (ES-009-B)
ACCUTASE™ cell detachment solution, Stemcell Technologies, #07920, 100 mL
mTeSR®1, StemCell Technologies, #05850 1 Kit , defined human pluripotent stem cell liquid media
Y-27632 dihydrochloride monohydrate ((R)-(+)-trans-4-(1-Aminoethyl)-N-(4-Pyridyl)cyclohexanecarboxamide dihydrochloride, Tocris,
Advanced RPMI Medium 1640 (1×), liquid, No Glutamine Phenol Red 500 ml,
Invitrogen, #12633-012, basel cell growth media
Glutamine 100×#25030-081 from Invitrogen
Image-iT™ FX Signal Enhancer, Invitrogen, #I36933 (optional), fluorescence stain signal enhancer
Triton X-100, Sigma, #T8787
SlowFade® Gold antifade reagent with DAPI, Invitrogen, #S36938 fluorescence stain signal preservative
Dimethyl sulfoxide (DMSO), #494429—Sigma
DE diff medium-I (MAKE afresh on the same day):
   Advanced RPMI Medium 1640
   Glutamine (1×, add fresh)
   human Activin A 100 ng/ml
   human Wnt3a 25 ng/ml.
DE diff medium-II (MAKE afresh on the same day):
   Advanced RPMI Medium 1640
   Glutamine (1×, add fresh)
   human Activin A 100 ng/ml
   0.1% Fetal Bovine Serum (FBS).
Matrtigel coating of tissue culture plates:
Coat 96 well with Matrigel using a 1:80 dilution.

### Protocol Methods

### Pluripotent Stem Cell Preparation:

Cell Culture. H9 human ES cells (From WiCell) were thawed in mTeSR medium with 10 uM Y-27632. Plate 3 millions cells in two 100 mm tissue culture grade dishes pre-coated with ES cell qualified Matrigel (BD Biosciences). Y-27632 is required only during seeding and it should be removed within 24 hours after seeding. Change mTeSR media every day. Cells should be near-confluent within 3-4 days.

### Accutase and Seed on 96 Well Plates

When cells become about 50% confluent remove media, rinse with PBS, then add 2 ml of accutase per 100 mm plate. Cells should detach within 3 minutes Immediately add 8 ml mTeSR with 10 uM Y-27632, transfer 20 ml (from 2×100 mm plate) to 50 ml tubes. Spin at 400×g for 6 min. Remove supernatant. Add 10 ml mTeSR with Y-27632. Pipet up and down to suspend cell pellet. Count the number of viable cells and re-suspend to 1 millions cells/ml. Seed cells at between 5000-50,000 cells/96 well. Depending on the specific cell line and lot of cells, this number may vary in order to attain optimal cell density. For these experiments, 15,000 cells/well were plated. Change mTESR media every day for 2-5 days.

### Pluripotent Stem Cell Directed Differentiation and Analysis

Day 0. START DIFFERENTIATION with Diff Media-I with Drug

Cells should be evenly distributed in 96 well plates. Begin differentiation when cells are about 35% confluency in the wells of 96 well plate (after 2-5 days). Remove media, and wash it with Advanced RPMI 4-5 times. It's important to wash away mTESR completely, as it contains very high concentration of various factors that maintain the cells in the undifferentiated state. Add DE diff medium-I with or without test compound. Vortex plate at 500 rpm for 3 min. Leave the plate for 24 hours.

Day 1. Change to Diff Media-II with Drug

Wash plates three times with Advance RPMI quickly (˜10 min) Add DE diff medium-II with or without compound. Vortex plate at 500 rpm for 3 min. Leave the plate for 48 hours. Note that cells were treated with drugs for a total of 72 hours.

Day 3. STOP AND FIX CELLS

1. Remove medium from 96 well plate. Wash well with DPBS GENTLY three times to remove debris or dead cells on top. Add freshly prepared 3%

Formaldehyde in PBS which was pre-warmed at 37° C. Add this on cells for 15 min Long fixation may cause high background or antigen alteration. Wash with PBS three times to remove PBS.

2. Permeabilize cells with PBS with 0.1% TritonX-100 for 15 min

3. Apply 30 ul ImageIT-FX solution per 96 well. Vortex at 700 rpm for 1 min Incubate for 30 minutes at room temperature.

4. Wash with Blocking Buffer (PBS with 10% donkey serum, 0.3% Triton X-100, 1% BSA) once, then add Blocking Buffer, leave 20 min

5. Add Sox17 antibody (1:10 dilution) in Blocking Buffer. Incubate for 3 hours at room temp.

6. Wash with PBS with 1% BSA four times.

7. Add PBS with 1% BSA with DAPI or Hoechst 33242 (5 ug/ml final conc for both) for 5 min

If you use SlowFade with DAPI later, this procedure is not needed.

8. Add SlowFade with DAPI 30 ul to each 96 well.

### Data Analysis

Measure the fluorescent intensity at 557 nm by Envision Plate reader.

### Protocol and Performance Criteria

The preceding example illustrates the invention as applied to the human embryonic stem cell H9 (Wisconsin Alumi Research Foundation, WARF). When applying the method to other pluripotent stem cell lines, one must determine the typical variables effect line-dependent differentiation efficiency such as initial cell seeding density, the concentrations of growth factors in the media and the duration of the differentiation. During this optimization process described above, we monitored the efficiency of mesendoderm formation as well as the drug effect mediated by thalidomide to identify the optimal conditions for the prediction of teratogenic risk.

The foregoing invention has been described in some detail by way of illustration and example, for purposes of clarity and understanding. Therefore, it is to be understood that the above description is intended to be illustrative and not restrictive. All patents, patent applications, and publications cited in this application are hereby incorporated by reference in their entirety for all purposes.

1. A method of assessing the teratogenic risk of a compound, said method comprising:
(1) contacting the compound with pluripotent stem cells that are being differentiated into mesendoderm;
(2) measuring the protein expression of one or more mesendodermal markers selected from the group consisting of Sox17, EOMES, T-brachyury or combinations thereof;
(3) determining the 1050 concentration of the compound which results in 50% inhibition of the protein expression of said one or more markers;
(4) comparing the 1050 concentration determined in step (3) with the IC50 concentrations of: (a) one or more compounds with known teratogenic risks and (b) one or more compounds with known non-teratogenic risks, to establish a teratogenic risk of the compound being assessed;
(5) adjusting the dose and administration of the compound to be provided to a patient based on the teratogenic risk measurement.

2. The method according to claim 1, wherein the pluripotent stem cells are of primate origin.

3. The method according to claim 1, wherein the pluripotent stem cells are embryonic stem cells.

4-21. (canceled)

22. The method according to claim 2, wherein the pluripotent stem cells are embryonic stem cells.

23. The method according to claim 1, wherein the pluripotent stem cells are H9 human embryonic stem cells.

24. The method according to claim 2, wherein the pluripotent stem cells are H9 human embryonic stem cells.

25. The method according to claim 3, wherein the pluripotent stem cells are H9 human embryonic stem cells.

26. The method according to claim 22, wherein the pluripotent stem cells are H9 human embryonic stem cells.

27. The method according to claim 1, wherein the pluripotent stem cells are induced pluripotent stem cells.

28. The method according to claim 2, wherein the pluripotent stem cells are induced pluripotent stem cells.

29. The method according to claim 3, wherein the pluripotent stem cells are induced pluripotent stem cells.

30. The method according to claim 1, wherein at least one of the mesendodermal markers is selected from the group SOX17, is EOMES, T-brachyury, or combinations thereof.

31. The method according to claim 2, wherein at least one of the mesendodermal markers is selected from the group SOX17, is EOMES, T-brachyury, or combinations thereof.

32. The method according to claim 3, wherein at least one of the mesendodermal markers is selected from the group SOX17, is EOMES, T-brachyury, or combinations thereof.

33. The method according to claim 22, wherein at least one of the mesendodermal markers is selected from the group SOX17, is EOMES, T-brachyury, or combinations thereof.

34. The method according to claim 23, wherein at least one of the mesendodermal markers is selected from the group SOX17, is EOMES, T-brachyury, or combinations thereof.

35. The method according to claim 24, wherein at least one of the mesendodermal markers is selected from the group SOX17, is EOMES, T-brachyury, or combinations thereof.

36. The method according to claim 25, wherein at least one of the mesendodermal markers is selected from the group SOX17, is EOMES, T-brachyury, or combinations thereof.

37. The method according to claim 26, wherein at least one of the mesendodermal markers is selected from the group SOX17, is EOMES, T-brachyury, or combinations thereof.

38. The method according to claim 27, wherein at least one of the mesendodermal markers is selected from the group SOX17, is EOMES, T-brachyury, or combinations thereof.

39. The method according to claim 28, wherein the protein expression of SOX17 is the only mesendodermal marker measured.

40. The method according to claim 29, wherein the protein expression of SOX17 is the only mesendodermal marker measured.

41. The method according to claim 1, wherein the protein expression of SOX17 and EOMES are the only mesendodermal markers measured.

42. The method according to claim 1, wherein the protein expression of SOX17 and T-brachyury are the only mesendodermal markers measured.

43. The method according to claim 1, wherein the protein expression is selected from the group that measures by immuno-histochemistry, by flow cytometry, by fluorescence microscopy, or combinations thereof.

44. The method according to claim 1, wherein prior to step (1) about 40-60% of said pluripotent stem cells are differentiated into mesendoderm.

45. The method according to claim 1, wherein the one or more compounds with known teratogenic risks in step (4) are selected from the group consisting of actinomycin D, tretinoin, cytarabine, nocodazole, rotenone, tretinoin, isotretinoin, bromodeoxyuridine, doxorubicin, dorsomorphin, thalidomide, 5-Fluorouracil, dasatinib, sorafenib, valproic acid, sunitinib, ziprasidone, mianserine, vandetanib, diethylstilbestrol, 6-amino-nicotinamide, ritanserin, and gefitinib.

46. The method according to claim 1, wherein the one or more compounds with known teratogenic risks in step (4) are selected from the group consisting of thalidomide, mianserine, and ritanserin.

47. The method according to claim 1, wherein the one or more compounds with known non-teratogenic risks in step (4) are selected from the group consisting of esomeprazole, folate, catechin, lisuride, niacin, aspirin, ibuprofen, acyclovir, ketanserin, streptomycin, methyldopa, saccharin, caffeine, penicillin, and tegaserod.

48. The method according to claim 1, wherein the one or more compounds with known non-teratogenic risks in step (4) are selected from the group consisting of folate and methyldopa.

49. The method according to claim 1, wherein the one or more compounds with known teratogenic risks in step (4) are selected from the group consisting of: thalidomide, mianserine, and ritanserin; and the one or more compounds with known non-teratogenic risks are selected from the group consisting of folate and methyldopa.
